(19) United States
(12) Patent Application Publication
(10) Pub. No.: US 2003/0215533 A1
(43) Pub. Date: Nov. 20, 2003

(54) Method of preventing respiratory infections

(76) Inventor: Saeed Rezakhany, San Jose, CA (US)

Correspondence Address: Saeed Rezakhany, 6933 Melvin Dr, San Jose, CA, 95129, US

(21) Appl. No.: 10/411,965

(22) Filed: Apr. 10, 2003

Related U.S. Application Data
(60) Provisional application No. 60/371,882, filed on Apr. 11, 2002.

(51) Int. Cl. A61K 7/16; A61K 35/78

(52) U.S. Cl. 424/742; 514/164; 424/49

(57) ABSTRACT

A method is presented for prevention of various respiratory infections which can be caused by one or more pathogens (bacteria, viruses, and fungi) including, but not limited to, those causing the various forms of upper respiratory tract infection (common cold), influenza, bronchitis, laryngitis, etc. in human being, by using a non-toxic, easily accessible to public “effective formula” to attack the newly activated or the newly invading pathogen and rendering it ineffective. If the infection has already fully taken effect, the present method helps reduce the symptoms of the infection (illness) and its duration by preventing occurrence of secondary infections. This method may also be applied to other mammals.

This application claims benefit of provisional application No. 60/371,882, filed Apr. 11, 2002, the entire disclosure of which is considered to be part of the disclosure of this application and is hereby incorporated by reference.

### BACKGROUND OF THE INVENTION

Respiratory infections, particularly upper respiratory tract infections are very common and cause substantial suffering and hundreds of millions of dollars of economic loss every year.

The majority of the pathogens contributing to upper respiratory tract infections are spread through air and through touching of the infected surfaces and then touching one's eyes or nose.

The nasopharynx, the nasal passages, and the sinus cavities all play an important role in filtering and housing majority of these pathogens.

Prior to this invention, as of the date of writing of this application, no effective, easy to use, and widely accepted cure, treatment, remedy, and particularly no prevention had been found for the upper respiratory tract infections.

Human body is equipped with organs, such as the tonsils and the nervous system, that give early warnings about the invasion of the respiratory tract's lining by pathogens. This early warning mechanism is part of the effective method of preventing respiratory infections, presented in this patent application.

### SUMMARY OF THE INVENTION

The inventor has experienced that if the early warnings given by the various organs of the body, such as tonsils and the nervous system, are heeded and acted upon by taking an action to render the pathogen(s) causing the symptoms ineffective, the cold or flue will never fully develop and thus the major undesirable symptoms and the accompanying economic losses can be prevented.

The inventor has further experienced that the pathogens responsible for the common upper respiratory tract infections, i.e. common “cold and flue”, seem to be most vulnerable when they have been newly activated and/or they have newly invaded the body. This is the corner stone of this invention. That is, if the pathogen is attacked shortly after it invades the body it can easily be rendered ineffective.

Thus, based on the above two experiences, a method is presented here for prevention of various respiratory infections which can be caused by one or more pathogens (bacteria, viruses, and fungi) including, but not limited to, those causing the various forms of upper respiratory tract infection (common cold), influenza, bronchitis, laryngitis, etc. in human being, by using a non-toxic, easily accessible to public “effective formula” to attack the newly activated or the newly invading pathogen and rendering it ineffective. If the infection has already fully taken effect, the present method helps reduce the symptoms of the infection (illness) and its duration by preventing occurrence of secondary infections. This method may also be applied to other mammals.

### DETAILED DESCRIPTION OF THE INVENTION

Through experience, I have observed that the minor discomforts (symptoms) such as minor throat irritation (ache, itching, scratchiness), minor headache, minor chills or feeling abnormally hot without any physical efforts, minor eye irritations, during the cold season, in a normal, otherwise healthy person, are all directly related to the onset of one of the multiple kinds of the upper respiratory tract infections (“cold” or “flue”). Again I have experienced that these symptoms, however minor, must be taken seriously, especially during the cold season. I theorize that these symptoms, in an otherwise healthy person, indicate the onset of activation and/or invasion of the pathogens. These pathogens either already exist in the body of the person feeling the symptoms, or they are newly invading it.

Once even if one of the above symptoms is felt, the person must right away (experience showed “right away” safely can extend about one hour) implement (undertake) the method of this invention. Doing so will render the responsible pathogen(s) ineffective and thus prevent a full upper respiratory tract infection with its quite painful, uncomfortable, and costly consequences. That is the inventor has experienced that the pathogens responsible for “cold and flue” seem to be most vulnerable when they have been newly activated and/or they have newly invaded the body. This is the corner stone of this invention. This implies, if the pathogen is attacked shortly after it invades the body it can easily be rendered ineffective.

The method of this invention was found to be fully effective in every case that it was tried based on the guidelines presented here. For a few examples please refer to the examples section below. It was observed experimentally that, if the method presented here is not implemented shortly after the initial feeling of any of the above symptoms, the infection takes effect and develops into a major sickness with full set of symptoms.

The method presented here is implemented by using (without limiting the scope of this invention) one type of oral rinse (mouthwash) that is available in pharmacies without prescription of a doctor. This type of mouthwash typically has an ingredient list of: Active Ingredients—Thymol 0.064%, Eucalyptol 0.092%, Methyl Salicylate 0.060%, and Menthol 0.042%; Inactive Ingredients—Water, Alcohol 26.9%, Benzoic Acid, Poloxamer 407 and Caramel.

Right after initially feeling one or more of the above symptoms, about three tea spoons (15 milliliters) of the above liquid oral rinse (mouthwash) is poured into the mouth and by tilting the head backward it is guided to the throat area. Then gargling is started. The gargling should continue for approximately 40 seconds. During these 40 seconds the gargling should be stopped at least three times, and each time the vapors of the oral rinse from the throat area should be passed (gently forced) along with the exhaled air thorough the nasopharynx and the nasal passages to outside. Also while the air is inhaled, some of the vapor from the oral rinse is naturally passed along with the inhaled air through the larynx, over the vocal cords, and through trachea to the lungs. Also during this 40 seconds the mouthwash fluid should be swished around the mouth several times to thoroughly disinfect the mouth. At the end of the 40 seconds the mouthwash should be emptied out from the mouth. Then the mouth should be rinsed with water so that none or minimal amount of the mouthwash remains in the mouth. If one or both of the nasal passages are congested and thus they do not conduct air easily, then this method should be repeated when the passages open up or become less congested.

Upon feeling any of the minor symptoms discussed above, the gargling method just described should be done at least four times: morning, noon, evening, and night. This minimum frequency is for when no other infected people, such as family members, coworkers, classmates, etc., with developed symptoms are around. If such people also exist, or if a major upper respiratory tract infection epidemic is going on, then this gargling method should be repeated approximately every two hours from right after getting up, to bed time.

During the “cold and flue” eason, even if no symptoms exist, as a precaution and as a preventive mechanism, the method described above should be undertaken with frequency reduced to two times, once in the morning right after rising, and once before sleeping at night. Also, during the cold and flue season, within one hour of a meeting with people, the gargling method described above should be done.

It should be noted that the 15 ml of mouthwash and 40 seconds duration of gargling are the amount and duration suitable for a healthy adult of medium weight. The amount and duration vary with the age and weight of the person using the method of this invention, as well as with other factors such as tolerance for the “effective formula”. These various amounts and durations need to be determined through research and they need to be tabulated for various ages, weights, and other factors.

Also it should be noted that in case the respiratory infection has already entered its developed stages, then the method of this invention still is helpful by preventing and or minimizing the secondary infections. Thus, the person who has developed the full symptoms of the common cold or influenza, should implement the more frequent (that is every two hours) gargling method described above.

Detailed research is necessary to come up with a better “effective formula” than the example given in this application (i.e. the oral rinse), in terms of suitability for age, health, handicap, or other factors such as tolerance for taste, odor, etc. I theorize that such better formulae are quite possible. They can be in various forms: different oral rinse, vapors, sprays, injections, tablets, etc., which each could be suitable for a given category of end users such as human beings (children, adults, young, old, male, female) and other mammals.

Also research is necessary to reduce the side effects of the “effective formulae”. In the case of the oral rinse, the side effects, although not harmful and quite tolerable, never the less did exist and included bad taste, odor, burning sensation of throat, and the pharynx and the nasopharynx.

It should be noted that the method of this invention should not be used as a substitution for other established helpful procedures for good health and prevention of respiratory infections. These other procedures include: often washing hands thoroughly with soap and water; washing face, nose, eyes; not touching dirty hands to the eyes, nose, ears, mouth, and face; resting well, eating well, and exercising. Rather, the method presented here should be used in conjunction with these other good health procedures.

Finally, although the above discussion has been primarily regarding the common upper respiratory tract infections, I theorize that the above discussion holds true regarding most, if not all, other types of respiratory infections. Also the method presented here is applicable to other mammals as well, with a suitable “effective formula” administered on a regular periodic basis during the epidemic breakouts and/or during the season for a particular respiratory infection.

### EXAMPLES

### Example 1

A 44 years old male had experienced minor throat irritation. The method of this invention was applied as described in this application. The symptoms went away and no “cold or flue” developed.

### Example 2

A 77 years old male had experienced some “early warning symptoms” as described in this application, he utilized the method of this invention. Again no cold or flue developed.

### Example 3

An 11 years old female had experienced some of the minor symptoms described here, she implemented the method of this invention, again as a result no cold or flue developed.

### Example 4

A couple both had similar minor symptoms as described in this patent application. The husband did follow the guidelines for implementing the method of this invention, and the wife didn't. The husband did not develop any cold or flue, while the wife developed a major case of influenza.

### Example 5

A five years old male was experiencing some of the minor symptoms described above, he was helped to implement the method of this invention, he did not develop a cold or flue.

I claim:

1- A method for rendering ineffective a pathogen (bacterium, virus, fungus) which has newly invaded the respiratory tract of a mammal, specifically a human being, by administering a non-toxic composition (effective formula) to the mammal at specific times, thereby preventing the pathogen from fully infecting the mammal and causing the mammal to develop the full symptoms of the infection.

2- The method of claim 1, wherein the time of starting the administration of the effective formula is of great significance (without limiting the scope of this invention this “time of starting the administration” for human beings experiencing minor symptoms due to the onset of upper respiratory tract infections, was through experience determined to be approximately one hour).

3- The method of claim 1, wherein the effective formula is administered to an individual shortly after initially the individual feels any of the following “minor” symptoms which are not generally attributable to allergy and/or other disorders, and which are generally associated with early warning signs of the onset of a respiratory infection:
a) Minor head, eye, ear, throat, or body ache
b) Minor congestion, stuffy nose
c) Minor Cough
d) Itching in the throat, ear, or throat area
e) “Scratchy” throat, requiring clearing of phlegm (minor hoarseness)
f) “Scratchy” eyes, feeling roughness when moving eyes or when blinking
g) Sneezing
h) Feeling abnormally cold (chills and shivers), or abnormally hot under normal room temperatures.

4- The method of claim 1, wherein the effective formula is administered at periodic intervals regardless of any symptoms, after the suspicion of being newly infected or when the chances of being infected is good such as during times (seasons) when the spread of the respiratory infections is well known, after attending any meeting or gathering, and during an epidemic outbreak of a respiratory infectious illness.

5- The method of claim 1, wherein the duration between the initial symptoms of a suspected respiratory infection and the time of starting the method of this invention, the period between the successive administrations of the effective formula, and the amount of the formula to be used are all functions of the type of the respiratory infection, the effective formula's effectiveness and other properties, and the mammal using the formula.

6- The method of claim 1, wherein the composition to be administered comprises an oral rinse form of an effective formula.

7- The method of claim 6, wherein one of the effective formulae, without limiting the scope of this invention, was found by the inventor to be a common oral rinse (mouth wash) available over the counter (without prescription of a physician), which typically has an ingredient list of
(a) Active Ingredients—
I-Thymol 0.064%
II-Eucalyptol 0.092%
III-Methyl Salicylate 0.060%
IV-Menthol 0.042%
(b) Inactive Ingredients—
I-Water
II-Alcohol 26.9%
III-Benzoic Acid
IV-Poloxamer 407
V-Caramel.

8- The method of claim 7, wherein approximately three tea spoons (15 milliliters) of the composition (above liquid oral rinse or mouthwash) is poured into the mouth and by tilting the head backward it is guided to the throat area and
(a) gargling is started and continued for approximately 40 seconds
(b) at least three times during these 40 seconds the gargling should be stopped, and each time the vapors of the oral rinse from the throat area should be passed (gently forced) along with the exhaled air thorough the nasopharynx and the nasal passages to outside
(c) while the air is inhaled, some of the vapor from the oral rinse is naturally passed along with the inhaled air through the larynx, over the vocal cords, and through trachea to the lungs
(d) during this period of forty seconds the mouthwash fluid should be swished around the mouth thoroughly several times to disinfect the mouth
(e) at the end of the 40 seconds the mouthwash should be emptied out from the mouth and the mouth should be rinsed with water so that none or minimal amount of the mouthwash remains in the mouth
(f) if one or both of the nasal passages are congested and thus they do not conduct air easily, then this method should be repeated when the passages open up or become less congested

9- The method of claim 1, wherein the composition to be administered comprises a vapor form of an effective formula.

10- The method of claim 1, wherein the composition to be administered comprises a tablet form of an effective formula to be taken orally.

11- The method of claim 1, wherein the composition to be administered comprises a liquid form of an effective formula to be taken orally.

12- The method of claim 1, wherein the composition to be administered comprises an injection form of an effective formula.

13- The method of claim 1, wherein the composition to be administered comprises a nose drop form of an effective formula.

14- The method of claim 1, wherein the composition to be administered comprises a spray form of an effective formula.
